(19) United States
(12) Patent Application Publication
(10) Pub. No.: US 2015/0133537 A1
(43) Pub. Date: May 14, 2015

(54) 3 - (1' - ADAMANTYL) - 1 - AMINOMETHYL - 3, 4 - DIHYDRO - 5, 6 - DIHYDROXY - 1H - 2 - BENZOPYRAN FOR USE IN THE TREATMENT OF A DISEASE ASSOCIATED WITH BETA-AMYLOID INDUCED TOXICITY

(71) Applicant: The University of Manchester, Manchester (GB)

(72) Inventors: Farid Khan, Altrincham (GB); Andrew James Doig, Huddersfield (GB); Swananda R. Modak, Dombivli (E) (IN)

(73) Assignee: The University of Manchester, Manchester (GB)

(21) Appl. No.: 14/395,409

(22) PCT Filed: Apr. 18, 2013
(86) PCT No.: PCT/GB2013/050985

§ 371 Date: Oct. 17, 2014

(30) Foreign Application Priority Data
Apr. 20, 2012 (GB) 1206984.5

(51) Int. Cl. A61K 31/352

(52) U.S. Cl. CPC A61K 31/352
USPC 514/456; 549/406; 435/375

(57) ABSTRACT

The present invention relates to the new use of the compound 3-(1′-adamantyl)-1-aminomethyl-3,4-dihydro-5,6-dihydroxy-1H-2-benzopyran for the treatment of diseases or conditions that are associated with β-amyloid induced toxicity, such as Alzheimer's disease.

### FIELD OF THE INVENTION

The present invention relates to a new therapeutic use of a known compound. More specifically, the present invention relates to the use of a known compound for the treatment of diseases or conditions that are associated with β-amyloid induced toxicity, such as Alzheimer's disease.

### BACKGROUND OF THE INVENTION

β-amyloid (Aβ) is a peptide comprising 39-43 amino acids that is produced by the endoproteolysis of the amyloid precursor protein (APP). APP is first cleaved by β-secretase to give the membrane bound C99 peptide and then by γ-secretase to give Aβ.

Aβ is most commonly known clinically for its association with Alzheimer's disease. Alzheimer's disease is characterised pathologically by abnormally high levels of brain lesions (senile plaques) and neurofibrillary tangles in dead and dying neurons, and by elevated numbers of amyloid deposits in the walls of cerebral blood vessels. The major component of senile plaques is the Aβ protein, which readily self-assembles into amyloid fibrils. Longer variants of Aβ are more prone to form amyloid. Compelling evidence indicates that factors that increase the production of Aβ, particularly its more amyloidogenic variants, or that facilitate deposition or inhibit elimination of amyloid deposits, cause Alzheimer's or are risk factors for the disease¹.

β-Amyloid aggregation and deposition has also been implicated in other diseases or conditions such as inclusion body myositis² and vascular dementia³ and cerebral amyloid angiopathy.

In vitro and in vivo evidence has shown that it is soluble, oligomeric forms of Aβ that have potent neurotoxic activity and are the primary causes of neuronal injury and cell death occurring in Alzheimer's disease⁴⁻⁸. It has therefore been proposed that the primary target of a Aβ based therapy should therefore be Aβ oligomers, rather than other less, or non-toxic, species.

There is therefore a need for new therapies capable of lowering or eliminating Aβ induced toxicity.

### SUMMARY OF THE INVENTION

The present invention resides in the recognition that the compound 3-(1′-adamantyl)-1-aminomethyl-3,4-dihydro-5,6-dihydroxy-1H-2-benzopyran is a potent inhibitor of β-amyloid induced toxicity.

A particular form of this compound, (1R,3S) 3-(1′-adamantyl)-1-aminomethyl-3,4-dihydro-5,6-dihydroxy-1H-2-benzopyran hydrochloride, is known in the art to be a dopamine 1 (D1) receptor agonist, called “A-77636”. A-77636 has been shown to have activity against Parkinson's disease in animal models⁹ and it has also been suggested to treat cocaine addiction¹⁰. A-77636 is also known to cross the blood-brain barrier, a crucial requirement for an Alzheimer's drug¹⁶.

Therefore, in a first aspect, the present invention provides 3-(1′-adamantyl)-1-aminomethyl-3,4-dihydro-5,6-dihydroxy-1H-2-benzopyran, or a pharmaceutically acceptable salt or solvate thereof, for use in the treatment of a disease or condition associated with β-amyloid induced toxicity.

In a further aspect the present invention provides a pharmaceutical composition comprising 3-(1′-adamantyl)-1-aminomethyl-3,4-dihydro-5,6-dihydroxy-1H-2-benzopyran, or a pharmaceutically acceptable salt or solvate thereof, and one or more pharmaceutically acceptable excipients, for use in the treatment of a disease or condition associated with β-amyloid induced toxicity.

In a further aspect, the present invention provides the use of 3-(1′-adamantyl)-1-aminomethyl-3,4-dihydro-5,6-dihydroxy-1H-2-benzopyran, or a pharmaceutically acceptable salt or solvate thereof, in the manufacture of a medicament for use in the treatment of a disease or condition associated with β-amyloid induced toxicity.

In another aspect, the present invention provides a method of treating a disease or condition associated with β-amyloid induced toxicity, said method comprising administering to a subject in need of such treatment a therapeutically effective amount of 3-(1′-adamantyl)-1-aminomethyl-3,4-dihydro-5,6-dihydroxy-1H-2-benzopyran, or a pharmaceutically acceptable salt or solvate thereof.

In another aspect, the present invention provides a method of treating a disease or condition associated with β-amyloid induced toxicity, said method comprising administering to a subject in need of such treatment a therapeutically effective amount of a pharmaceutical composition comprising 3-(1′-adamantyl)-1-aminomethyl-3,4-dihydro-5,6-dihydroxy-1H-2-benzopyran, or a pharmaceutically acceptable salt or solvate thereof, and one or more pharmaceutically acceptable excipients.

In a further aspect, the present invention provides 3-(1′-adamantyl)-1-aminomethyl-3,4-dihydro-5,6-dihydroxy-1H-2-benzopyran, or a pharmaceutically acceptable salt or solvate thereof, for use in the inhibition of β-amyloid induced toxicity.

In a further aspect, the present invention provides a method of inhibiting β-amyloid induced toxicity (in vitro or in vivo), said method comprising administering an effective amount of 3-(1′-adamantyl)-1-aminomethyl-3,4-dihydro-5,6-dihydroxy-1H-2-benzopyran, or a pharmaceutically acceptable salt or solvate thereof.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a disease or condition. “Treating” or “treatment” therefore includes: (1) preventing or delaying the appearance of clinical symptoms of the disease or condition developing in a subject that may be afflicted with or predisposed to the disease or condition, but does not yet experience or display clinical or subclinical symptoms of the disease or condition, (2) inhibiting the disease or condition, i.e., arresting, reducing or delaying the development of the disease or condition or a relapse thereof (in case of maintenance treatment) or at least one clinical or subclinical symptom thereof, or (3) relieving or attenuating the disease or condition, i.e., causing regression of the disease or condition or at least one of its clinical or subclinical symptoms.

A “therapeutically effective amount” means the amount of the compound that, when administered to a subject for treating a disease or condition referred to herein, is sufficient to effect such treatment for the disease or condition. The “therapeutically effective amount” will vary depending on the form of the compound (e.g. the salt form), the disease or condition concerned and its severity, as well as the age, weight, etc., of the subject to be treated.

The term “subject” is used herein to mean a warm blooded mammal. Thus, the compound of the present invention may be used for human and/or veterinary applications. In a particular embodiment, the subject is a human.

### The Compound of the Invention

The compound of the present invention is 3-(1′-adamantyl)-1-aminomethyl-3,4-dihydro-5,6-dihydroxy-1H-2-benzopyran. The structure of this compound is shown below:

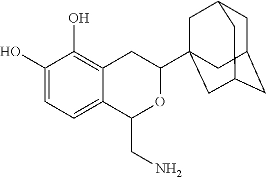 [SMILES: NCC1OC(C23CC4CC(CC(C4)C2)C3)Cc2c1ccc(O)c2O]

The compound of the invention may exist in one or more enantiomeric or diastereomeric forms. In particular, the compound of the invention can exist in the R or S configuration at positions 1 and 3 of the benzopyran ring.

The compound may exist as a single enantiomeric/diastereomeric form or as a mixture of enantiomeric/diastereomeric forms.

All enantiomeric/diastereomeric forms of the 3-(1′-adamantyl)-1-aminomethyl-3,4-dihydro-5,6-dihydroxy-1H-2-benzopyran compound that are capable of inhibiting β-amyloid induced toxicity are encompassed by the present invention.

In an embodiment, the compound is (1R,3S) 3-(1′-adamantyl)-1-aminomethyl-3,4-dihydro-5,6-dihydroxy-1H-2-benzopyran, or a pharmaceutically acceptable salt or solvate thereof. This compound has the following structure:

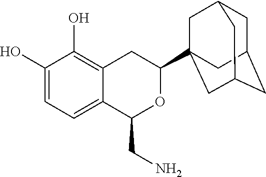 [SMILES: NC[C@@H]1O[C@H](C23CC4CC(CC(C4)C2)C3)Cc2c1ccc(O)c2O]

A suitable pharmaceutically acceptable salt of the compound of the invention is, for example, an acid-addition salt of the compound formed with an acid such as hydrochloric, hydrobromic, sulfuric, phosphoric, trifluoroacetic, formic, citric or maleic acid.

In a particular embodiment, the compound of the invention is in the form of a hydrochloride salt.

In a particular embodiment of the invention, the compound of the invention is (1R,3S) 3-(1′-adamantyl)-1-aminomethyl-3,4-dihydro-5,6-dihydroxy-1H-2-benzopyran hydrochloride.

It is also to be understood that the compound of the invention may also exist in solvated as well as unsolvated forms, such as, for example, hydrated forms. It is to be understood that the invention encompasses all such solvated forms that are capable of inhibiting β-amyloid induced toxicity.

It is also to be understood that the compound of the invention may also exhibit polymorphism, and that the invention encompasses all such polymorphic forms that are capable of inhibiting β-amyloid induced toxicity.

The compound of the invention may also be administered in the form of a pro-drug which is broken down in the human or animal body to release a compound of the invention. A pro-drug may be used to alter the physical properties and/or the pharmacokinetic properties of the compound of the invention. A pro-drug can be formed when the compound of the invention contains a suitable group or substituent to which a property-modifying group can be attached. Examples of pro-drugs include in vivo cleavable ester derivatives that may be formed at one of the hydroxy groups of the compound of the invention and/or in-vivo cleavable amide derivatives that may be formed at the amino group of the compound of the invention.

Accordingly, the present invention includes the compound of the invention as defined hereinbefore when made available within the human or animal body by way of cleavage of a pro-drug thereof. Accordingly, the present invention includes the compound of the invention being produced in the human or animal body by way of metabolism of a precursor compound that is the compound of the invention may be metabolically-produced.

A suitable pharmaceutically acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

The in vivo effects of the compound of the invention may be exerted in part by one or more metabolites that are formed within the human or animal body after administration of a compound of the invention. As stated hereinbefore, the in vivo effects of a compound of the invention may also be exerted by way of metabolism of a precursor compound (a pro-drug).

### Synthesis

The compound of the present invention can be sourced commercially and/or prepared by synthetic techniques known in the art.

### Pharmaceutical Compositions

According to a further aspect of the invention there is provided a pharmaceutical composition which comprises the compound of the invention as defined hereinbefore, or a pharmaceutically acceptable salt or solvate thereof, in association with a pharmaceutically acceptable diluent or carrier, for use in the treatment of a disease or condition associated with β-amyloid induced toxicity.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients well known in the art. Thus, compositions intended for oral use may contain, for example, one or more colouring, sweetening, flavouring and/or preservative agents.

The amount of active ingredient that is combined with one or more excipients to produce a single dosage form will necessarily vary depending upon the individual treated and the particular route of administration. For example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

Diseases or Conditions Associated with β-Amyloid Induced Toxicity

The biological assays described in the accompany examples demonstrate that the compound of the invention is a potent inhibitor of β-amyloid induced toxicity.

The compound of the invention is therefore suitable for the treatment (including prophylactic treatment) of a disease or condition associated with β-amyloid induced toxicity. Examples of such conditions include: Alzheimer's disease; inclusion body myositis² and vascular dementia³ and cerebral amyloid angiopathy.

In a particular embodiment, the compound of the invention can be used for the treatment (including prophylactic treatment) of Alzheimer's disease.

The compound of the invention is suitably administered in a therapeutically effective amount to a patient in need of treatment.

### Mechanism of Action

As previously stated, the compound of the invention is known to be a D1 receptor agonist. However, the mechanism by which the compound of the invention inhibits β-amyloid induced toxicity is not thought to be mediated by its D1 receptor agonist activity. Data for other D1 receptor agonists is presented in Example 5 herein and this data clearly shows that the inhibition of Aβ toxicity is not a general property of D1 dopamine receptor agonists.

The data presented in Example 4 herein indicates that one possible mechanism by which the compound of the invention may be acting is by binding with the RACK1 protein. RACK1 is known to modulate glutamatergic and dopaminergic neurotransmitter systems as well as helping with the maintenance of Ca²⁺ homeostasis, which appears to be disrupted as a result of Aβ42 aggregation^{18, 19, 21}. Down regulation of RACK1 is also a known phenomenon associated with AD²⁰. Therefore, and without wishing to be bound by any particular theory, it is possible that the compound of the invention is helping to restore the RACK1 level in the presence of β-amyloid and this may in turn indirectly improve the altered neurotransmitter systems associated with β-amyloid aggregation, and thereby possibly reducing the β-amyloid induced cytotoxicity observed.

### Routes of Administration

The compound of the invention or a pharmaceutical composition comprising this compound may be administered to a subject by any convenient route of administration. Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compound of the invention may be used as a sole therapy or may involve, in addition to the compound of the invention, therapy with one or more additional therapeutic agents.

Thus, in another aspect, the present invention provides the compound of the invention as defined herein, or a pharmaceutically acceptable salt or solvate thereof, for use in the treatment of a disease or condition associated with β-amyloid induced toxicity in combination with one or more additional therapeutic agents.

In another aspect, the present invention provides the use of the compound of the invention as defined herein, or a pharmaceutically acceptable salt or solvate thereof, in the manufacture of medicament for use in the treatment of a condition associated with β-amyloid induced toxicity in combination with one or more additional therapeutic agents.

Such conjoint treatment may be achieved by way of the simultaneous, sequential or separate dosing of the individual components of the treatment. Such combination products employ the compound of this invention within the dosage range described hereinbefore and the other pharmaceutically-active agent within its approved dosage range.

According to a further aspect of the invention there is provided a combination suitable for use in the treatment of β-amyloid induced toxicity comprising a compound of the invention as defined hereinbefore, or a pharmaceutically acceptable salt or solvate thereof, and one or more additional therapeutic agents.

Herein, where the term “combination” is used, it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises the compound of the invention, or a pharmaceutically acceptable salt or solvate thereof, one or more additional therapeutic agents, and a pharmaceutically acceptable diluent or carrier.

### BRIEF DESCRIPTION OF THE DRAWINGS

The invention is described further in reference to the accompanying Figures in which:

FIG. 1 shows the concentration dependent response of (1R,3S) 3-(1′-adamantyl)-1-aminomethyl-3,4-dihydro-5,6-dihydroxy-1H-2-benzopyran hydrochloride (A-77636) on extracellular Aβ42 aggregation using SHSY5Y cells; and

FIG. 2 shows the data shown in table 3 of Example 3 in graphical form.

FIG. 3 shows the in cell western assay for the analysis of RACK1 protein expression after Aβ42 and A-77636 treatment. SH-SY5Y cells were seeded for 1.5×10⁴/well. The cells were treated for two conditions, Aβ42 (1 μM) only and Aβ42 (1 μM):A-77636 (1 μM) for 24 hr. The parallel control cells were also treated with A-77636 (1 μM) only. (A) The in cell western assay demonstrated down regulation of RACK1 expression for the Aβ42 treatment by showing 55% decrease in RACK1 expression compared to the control cells. Administration of 1 μM A-77636 to the Aβ42 (1 μM) treated SH-SY5Y cells acts as a partial enhancer of RACK1 expression by showing 31% increase in the RACK1 levels when compared to Aβ42 (1 μM) only. (B) The difference in the levels of RACK1 expression for the SH-SY5Y cells treated with Aβ42 and A-77636 is observed through the Odyssey Infrared Imaging System captured at 700 nm by setting the intensity at 4.5. The data is represented as mean percentage fluorescence intensity which is proportional to RACK1 expression after normalization to loading control alpha tubulin, n=3, error bars represent standard error and p<0.05 when compared to the control cells (Aβ-ve). p>0.05 for A-77636 (1 μM) treated Aβ42-ve cells.

FIG. 4 shows the screening of further dopamine receptor agonists to identify hits for Aβ42 toxicity inhibitor. Nine compounds acting as dopamine receptor agonists having selectivity towards D1 receptors were screened on the SH-SY5Y cells treated with Aβ42 (1 μM) for 24 hr. The compound and target peptide were added simultaneously and incubated at 37° C. followed by cellular viability measurement using MTT assay. Compound A demonstrated partial inhibition of the Aβ42 toxicity by showing 81% cellular viability (p<0.05), whereas rest of the compounds were unable to rescue the SH-SY5Y cells from the extracellular Aβ42 toxicity. Data are represented as mean percentage viability, with no treatment controls set as 100%, n=3 and the error bars represent standard error.

### EXAMPLES

The invention will now be illustrated in the following Examples.

### Materials

(1R,3S) 3-(1′-adamantyl)-1-aminomethyl-3,4-dihydro-5,6-dihydroxy-1H-2-benzopyran hydrochloride (A-77636) was sourced from the commercially available Lopac compound library (a collection of 1280 pharmacologically active compounds that have highly diverse structures and modes-of-action). The Lopac library was provided as a gift from Lumophore Ltd. and originally purchased from Sigma Ltd.

The Aβ42 peptide was purchased from rPeptide (http://www.rpeptide.com/). SH-SY5Y cells were purchased from ATCC (http://www.atcc.org/ATCCAdvancedCatalogSearch/ProductDetails/tabid/452/Default.aspx?ATCCNum=CRL-2266&Template=cellBiology).

### Stock Preparation of Aβ Peptide:

Aβ42 was dissolved in high grade 100% 1,1,1,3,3,3-hexafluoroisopropanol (HFIP) (Sigma) followed by 3 cycles of vortexing 30 s each to maintain the peptide in monomeric state. This was followed by dissolution of the Aβ42 in an appropriate volume of HFIP to make 1 mM concentration and stored at 4° C. until required. The peptide was then dried in liquid N₂ and lyophilised overnight. The lyophilised form of Aβ42 was then sealed with parafilm and stored at −20° C. until required for further experimental assays.

### Example 1

### MTT Toxicity Assessment of SYSHY5Y Cell Viability in the Presence of Aβ42

The MTT toxicity assay is extensively used in studies measuring Aβ toxicity^{11, 12}.

### MTT Assay:

The MTT (3-(4,5-dimethylthiazol-2-yl)-2,5-diphenyltetrazolium bromide) assay was conducted on the SH-SY5Y cells to observe the effects of compound on the cells treated with extracellular Aβ42. The MTT assay provides a colorimetric analysis of cell viability by the conversion of MTT into formazan product by the mitochondrial enzyme succinate dehydrogenase. For cytotoxicity experiments cells were washed twice with Dulbecco's PBS followed by trypsinisation of cells, centrifugation at 1800 rpm and seeding the cells on 96 well flat bottom plates (Costar) in Opti-MEM media without phenol red supplemented with 1.5% FCS, 1% L-glutamine, 1% Penicillin-streptomycin (P/S) and 1% non essential amino acids (NEAA).

The cells were seeded at 1×10⁵/ml density in a 96 well flat bottom plate (Costar) and incubated for 24 hr in a humidified incubator at 37° C. with 5% CO₂. This was followed by removal of the media and adding fresh media to the cells containing 1 μM Aβ42 and 1 μM A-77636:

After 24 hr; 50 μl of the media was removed and 10 μl of MTT (2.5 mg/ml) was added to the cells. This was followed by incubation for further 4 hr at 37° C. and 5% CO₂. MTT reduction was characterized by adding 100 μl solubilization solution containing 50 ml isopropyl alcohol and 197 μl of 37% HCl. The cytotoxicity was determined by measuring the absorbance at 570 nm using Polarstar BMG labtech plate reader. The average viability for the cells without Aβ42 (healthy cells) was considered to be 100% for each experiment whereas 0.1% Triton X-100 was added to the live cells to produce 0% viable cells as a control for 100% nonviable or dead cells.

### Results

MTT results are shown in Table 1 below

TABLE 1

| | | Aβ42 +ve | A-77636 |
|---|---|---|---|
| | Aβ42 −ve | (1 μM) | (1 μM) |
| Experiment 1 | 100% | 65% | 82% |
| Experiment 2 | 100% | 63% | 69% |
| Experiment 3 | 100% | 68% | 92% |
| Average | 100% | 65% | 81% |
| Standard deviation | 0% | 2.5% | 11.5% |
| p value | 0 | | 0.009 |

These results demonstrate that the compound of the invention (A-77636) is capable of inhibiting the toxic effects of Aβ42 (when compared to Aβ42 with and without A-77636).

### Example 2

### MTT Toxicity Assessment of SYSHY5Y Cell Viability in the Presence of Aβ42 and Varying Concentrations of (1R,3S)3-(1′-adamantyl)-1-aminomethyl-3,4-dihydro-5,6-dihydroxy-1H-2-benzopyran hydrochloride (A-77636)

Compound A-77636 was screened at varying concentrations on the SH-SY5Y cells treated with extracellular Aβ42. For the MTT cytotoxicity assay, A-77636 was diluted in the Opti-MEM media supplemented with 1.5% FCS, 1% P/S, 1% L-glutamine and 1% NEAA. The target compound was added to the SH-SY5Y cells at concentrations of 100 μM, 10 μM, 1 μM, 100 nM, 10 nM and 1 nM.

The cells were seeded at 1×10⁵/ml density in a 96 well flat bottom plate (Costar) and incubated for 24 hr in a humidified incubator at 37° C. with 5% CO₂. This was followed by removal of the media and adding fresh media to the cells containing Aβ42 and A-77636. The final concentration of the target peptide and Compound A-77636 within the cells was at 1 μM:100 μM, 1 μM:10 μM, 1 μM:1 μM, 1 μM:100 nM, 1 μM:10 nM and 1 μM:1 nM. The cells were then incubated for 24 hr in the humidified incubator at 37° C. in 5% CO₂.

After 24 hr; 500 of the media was removed and 10 μl of MTT (2.5 mg/ml) was added to the cells. This was followed by incubation for further 4 hr at 37° C. and 5% CO₂. MTT reduction was characterized by adding 100 μl solubilization solution containing 50 ml isopropyl alcohol and 197 μl of 37% HCl. The 96 well plates were kept at room temperature for 6 hr to dissolve the formazan crystals. The cytotoxicity was determined by measuring the absorbance at 570 nm using Polarstar BMG labtech plate reader. The average viability for the cells without Aβ42 (healthy cells) was considered to be 100% for each experiment whereas 0.1% Triton X-100 was added to the live cells to produce 0% viable cells as a control for 100% nonviable or dead cells.

### Results

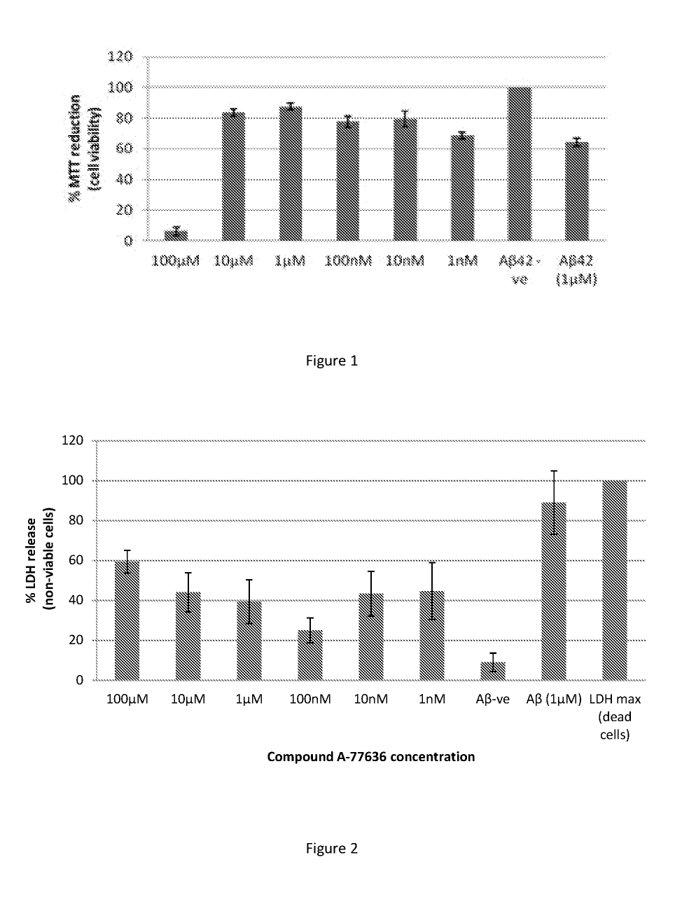

The MTT results are shown in Table 2 below and in FIG. 1.

TABLE 2
MTT cytotoxicty assay to observe the effects of varying
concentrations of Compound A-77636 on the SHSY5Y cells
treated with 1 μM Aβ42.

| Compound A-77636 | | | | | | | | |
|---|---|---|---|---|---|---|---|---|
| | 100 | 10 | 1 | 100 | 10 | 1 | | Aβ42 |
| | μM | μM | μM | nM | nM | nM | Aβ42-ve | (1 μM) |
| Exp 1 | 6 | 86 | 86 | 81 | 84 | 68 | 100 | 64 |
| Exp 2 | 9 | 84 | 87 | 78 | 80 | 71 | 100 | 67 |
| Exp 3 | 4 | 81 | 90 | 74 | 74 | 67 | 100 | 62 |
| Avg | 6 | 83 | 87 | 77 | 79 | 68 | 100 | 64 |
| Std | 2.5 | 2.5 | 2.1 | 3.5 | 5.0 | 2.1 | 0 | 2.5 |
| Dev | | | | | | | | |

The compound of the invention (A-77636) is toxic at 100 μM, active from 10 nM to 10 μM and inactive at 1 nM.

### Example 3

### LDH Toxicity Assessment of SYSHY5Y Cell Viability in the Presence of Aβ42 and Varying Concentrations of (1R,3S) 3-(1′-adamantyl)-1-aminomethyl-3,4-dihydro-5,6-dihydroxy-1H-2-benzopyran hydrochloride (A-77636)

### Concentration Dependent LDH Assay on the SHSY5Y Cells Using Compound A-77636:

The LDH (lactate dehydrogenase) assay was conducted on the SHSY5Y cells treated with Aβ42 (1 μM) using A-77636 at varying concentrations. LDH is a ubiquitously present cytoplasmic enzyme also found abundant in the neuronal cell lines¹⁴. The LDH assay provides the measure of cytotoxicity, wherein the amount of LDH released quantifies cell membrane damage, caused either by apoptosis or necrosis^{13,15}. The commercially available Cyto Tox-One assay (Promega; G7890) was used here which provides a fluorescent measure of LDH release, wherein the reduction of resazurin dye to resofurin is coupled through enzymatic conversion involving NADH. The generation of fluorescent dye resofurin is proportional to the amount of LDH release.

A-77636 was screened at varying concentrations on the SHSY5Y cells treated with extracellular Aβ42. For the LDH cytotoxicity assay, A-77636 was diluted in the Opti-MEM media supplemented with 1.5% FCS, 1% P/S, 1% L-glutamine and 1% NEAA. A-77636 was added to the SHSY5Y cells at concentrations of 100 μM, 10 μM, 1 μM, 100 nM, 10 nM and 1 nM.

The cells were seeded at 1×10⁵/ml density in a 96 well black, flat bottom plate (BD Biosciences) and incubated for 24 hr in a humidified incubator at 37° C. with 5% CO₂. This was followed by the removal of media and adding fresh media to the cells containing Aβ42:A77636. The final concentration of the target peptide and A-77636 within the cells was 1 μM:100 μM, 1 μM:10 μM, 1 μM:1 μM, 1 μM:100 nM, 1 μM:10 nM, and 1 μM:1 nM respectively. The cells were then incubated for 24 hr in the humidified incubator at 37° C. in 5% CO₂.

After 24 hr, the 96 well plates were removed from the incubator and equilibrated at 22° C. for 30 min. The Cyto Tox-One reagent was prepared according to the manufacturer's protocol by equilibrating the substrate mix at 22° C. and thawing the assay buffer at 37° C. in a water bath. This was followed by adding 11 ml of assay buffer to each vial of substrate mix. 100 μl of Cyto Tox-One reagent was added to each well. The preparation and addition of Cyto Tox-One reagent was undertaken in the dark to avoid increased background fluorescence and stored at −20° C. until required. The cells were further incubated at 22° C. for 10 min followed by addition of stop solution to the wells in the same order as that of the Cyto Tox-One reagent to avoid increased variation in the results. The stop solution stops formation of fluorescent product resofurin. The maximum LDH release (set as 100%) for each experiment was determined by adding 2 μl of lysis solution (9% triton X 100, Promega) to the cells prior to the addition of Cyto Tox-One reagent, providing a dead cell control. Cells without Aβ42 (healthy cells) were considered as a control for minimum LDH release or live cell control. The 96 well plates were then kept on the shaker for 10 sec and the fluorescence was measured immediately with an excitation at 560 nm and emission at 590 nm using a Polarstar BMG plate reader.

The LDH assay results are shown in Table 3 below and in FIG. 2.

TABLE 3
below shows the effects of varying concentrations of Compound A-77636
on SHSY5Y cells treated with 1 μM Aβ42 through LDH assay

| | 100 | 10 | 1 | 100 | 10 | 1 | Aβ- | Aβ | LDH max |
|---|---|---|---|---|---|---|---|---|---|
| | μM | μM | μM | nM | nM | nM | ve | (1 μM) | (dead cells) |
| Exp 1 | 61 | 33 | 27 | 20 | 35 | 35 | 8 | 95 | 100 |
| Exp 2 | 53 | 47 | 48 | 23 | 39 | 38 | 14 | 71 | 100 |
| Exp 3 | 64 | 52 | 43 | 32 | 56 | 61 | 5 | 101 | 100 |
| Avg | 59 | 44 | 39 | 25 | 43 | 45 | 9 | 89 | 100 |
| Std | 5.6 | 9.8 | 10.9 | 6.2 | 11.1 | 14.2 | 4.5 | 15.8 | 0 |
| dev | | | | | | | | | |

The SHSY5Y cells were treated with 1 μM Aβ42 and the effect of varying concentrations of Compound A-77636 was measured after 24 hr using Cyto Tox-One LDH assay. The results demonstrated 9% LDH release for Aβ-ve or healthy cells and 89% LDH release for the cells treated with 1 μM Aβ42 i.e. demonstrating 89% cytotoxicity when treated with extracellular Aβ42 (1 μM). A-77636 reduces Aβ42 toxicity at all concentrations from 100 μM to 1 nM. A-77636 is possibly toxic at 100 μM, as shown by increased LDH release (59%), though 100 μM A-77636 still reduces 1 μM Aβ42 toxicity, compare to 1 μM Aβ42 in isolation.

The compound of the invention (A-77636) is therefore a suitable candidate drug for the treatment of diseases of Alzheimer's disease and other diseases caused by Aβ toxicity.

### Example 4

### Differential Expression of RACK1 Protein in the Presence of Extracellular Aβ42 and A-77636 Treatment

### In Cell Western Assay:

The in cell western assay (ICW) is a cell based immunofluorescence technique which provides a rapid and sensitive measure of protein expression using microplates. The SH-SY5Y cells were treated with Aβ42 for the 24 hr duration using the microplates followed by fixation, immunostaining of the cells with primary antibody and fluorescently labelled secondary antibody followed by scanning the plate using the Odyssey Infrared Imaging System. This assay was undertaken to observe the expression of RACK1 protein using SH-SY5Y cells in the presence of Aβ42 and A-77636. Compound A-77636 belongs to the LOPAC library, which when administered to the extracellular Aβ42 treated SH-SY5Y cells demonstrated an inhibition of extracellular Aβ42 mediated cytotoxicity. The ICW assay demonstrated a differential expression of RACK1 for the Aβ42 (1 μM) treated and A-77636 (1 μM) treated SH-SY5Y cells, thereby helping to elucidate the plausible mode of action through which A-77636 might act in reducing the extracellular Aβ42 cytotoxicity.

### Methods:

### In Cell Western Assay for the Validation of RACK1 Expression:

The RACK1 expression was analyzed using the ICW assay on the SH-SY5Y cells treated with Aβ42 and A-77636. For the ICW assay 1.5×10⁴ cells per well were inoculated in Opti-MEM without phenol red supplemented with 1.5% FCS, 1% L-glutamine and 1% Penicillin Streptomycin using the 96 well clear flat bottom microplate (Costar). This was followed by removal of the media and adding fresh media containing Aβ42 (1 μM). In parallel cells were added with fresh media containing Aβ42 (1 μM) and compound A-77636 (1 μM), whereas only 1 μM A-77636 was added to the control cells. After further 24 hr, the media was discarded and cells were immediately fixed with 4% formaldehyde in PBS. The fixation was undertaken overnight at 4° C. The cells were washed 3 times with PBS and permeabilized by adding 2000 of 0.1% Triton X-100 in PBS. The cells were washed 3 times for 5 min each. This was followed by blocking the cells in Odyssey blocking buffer (LI-COR) for 1.5 hr at room temperature with moderate shaking on the plate shaker at 70 rpm. Rabbit antihuman RACK1 antibody (1:300) (ab72483) diluted in the Odyssey blocking buffer (LI-COR) was added to the cells followed by overnight incubation at 4° C. Alpha tubulin (1:200) (ab15246) was used as a loading control for these experiments. This was followed by washing the cells 3 times with 0.1% Tween 20 in PBS for 5 min each. The 500 of fluorescently labelled secondary antibody, antirabbit IRDye 680 LT (LI-COR) diluted at 1:800 in Odyssey blocking buffer were added to the cells followed by incubation for 1 hr at room temperature with gentle shaking. At this stage the cells were protected from light. The cells were then washed 3 times with 0.1% Tween 20 in PBS for 5 min each. After the final wash, the wash solution containing tween 20 was removed completely; the microplate was blotted gently on the paper towel and scanned immediately using an Odyssey Infrared scanner (LI-COR). The sensitivity of 4.5 was set for 700 nm channel for these experiments. The data was acquired by using Odyssey software, exported, analyzed in Excel and the values were background subtracted from the cells treated only with secondary antibody.

### Results:

### Differential Expression of RACK1 Protein in the Presence of Extracellular Aβ42 and A-77636 Treatment:

An ICW assay was undertaken on the SH-SY5Y cells treated with Aβ42 (1 μM) and A-77636 (1 μM) to determine the expression of RACK1 protein. RACK1 is known to be required for the activation and translocation of PKC¹⁷. Moreover RACK1 is also known to modulate glutamatergic and dopaminergic neurotransmitter systems as well as help in maintaining the Ca²⁺ homeostasis, which appears to be disrupted as a result of Aβ42 aggregation^{18, 19, 21}. Down regulation of RACK1 is a known phenomenon associated with AD²⁰. In this study differential expression of RACK1 protein was observed through the ICW assay for the cells treated with Aβ42 (1 μM) only and those with A-77636 (1 μM) treatment.

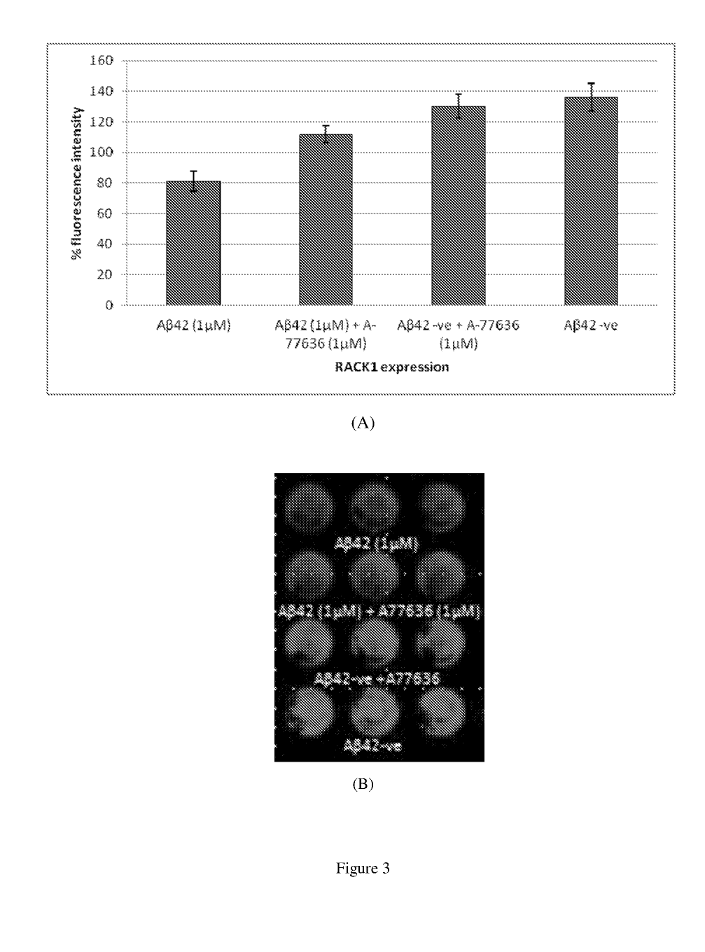

The RACK1 expression was measured for the SH-SY5Y cells treated with Aβ42 (1 μM) only, those treated with Aβ42:A-77636 at a final concentration of 1 μM:1 μM within the cells and A-77636 (1 μM) only for 24 hr. The results were compared with no treatment control cells. The 24 hr treatment of SH-SY5Y cells with 1 μM Aβ42 demonstrated decreased expression of RACK1 protein (81%) compared to the control cells (136%) with p<0.05, whereas 1 μM treatment with A-77636 for the Aβ42 (1 μM) treated SH-SY5Y cells demonstrated a partial improvement in the level of RACK1 to 112%, thereby demonstrating the possible mechanism of action though which A-77636 might help in improving the Aβ42 mediated cytotoxicity (FIG. 3). The ICW assay further helped in validating the results from MTT and LDH cytotoxicity assays, both of which have demonstrated the potential of A-77636 as a partial inhibitor of extracellular Aβ42 cytotoxicity. Moreover administration of 1 μM A-77636 alone to the control cells was unable to demonstrate any change in the RACK1 expression (130%) with p>0.05 when compared to the control cells. This illustrates the possible role of A-77636 in reducing the extracellular Aβ42 cytotoxicity, which might act by binding with the RACK1 protein and further helping in restoring the RACK1 level, thereby indirectly improving the altered neurotransmitter systems associated with Aβ42 aggregation possibly reducing Aβ42 cytotoxicity.

### Example 5

### The Effect of Alternative D1 Receptor Agonists on Aβ-Induced Toxicity

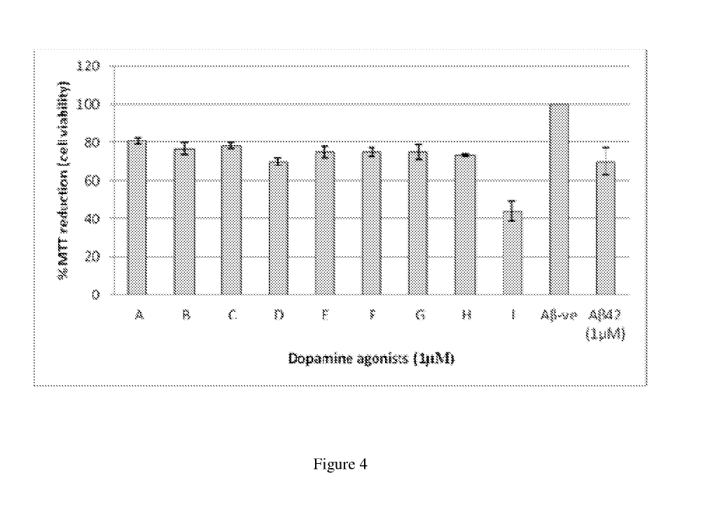

Further D1 dopamine receptor agonists (detailed in Table 4 below) were screened in order to determine whether they act as inhibitors of extracellular Aβ42 toxicity. These compounds were screened individually on the SH-SY5Y cells treated with Aβ42 (1 μM) to observe their effects on the Aβ42 cytotoxicity. Nine compounds in total were screened on the SH-SY5Y cells simultaneously with Aβ42 (1 μM) treatment. Compound A demonstrated partial inhibition of Aβ42 toxicity by illustrating 81% cell viability (p<0.05) whereas all the other compounds were unable to inhibit the Aβ42 toxicity demonstrated by statistically insignificant results (FIG. 4).

This data shows that inhibition of Aβ toxicity is not a general property of D1 dopamine receptor agonists.

TABLE 4
Properties of Dopamine receptor agonists shown in FIG. 4

| Compound | Name | Secondary Name | Selectivity | Description |
|---|---|---|---|---|
| A | R(+)-6-Bromo- | R(+)-6-Bromo-7,8-dihydroxy-3- | D1/D5 | D1 Dopamine |
| | APB | allyl-1-phenyl-2,3,4,5-tetrahydro- | | receptor agonist |
| | hydrobromide | 1H-3-benzazepine hydrobromide | | |
| B | Dihydrexidine | (±)-trans-10,11-Dihydroxy- | D1 | D1 dopamine |
| | hydrochloride | 5,6,6a,7,8,12b- | | receptor agonist |
| | | hexahydrobenzo[a]phenanthridine | | |
| | | hydrochloride | | |
| C | (±)-Chloro- | (±)-SKF-82958 hydrobromide | D1 | D1 dopamine |
| | APB | | | receptor agonist. |
| | hydrobromide | | | |
| D | (±)-SKF-38393 | (±)-1-Phenyl-2,3,4,5-tetrahydro- | D1 | D1 Dopamine |
| | hydrochloride | (1H)-3-benzazepine-7,8-diol | | receptor agonist |
| | | hydrochloride | | |
| E | Fenoldopam | SKF-82526 | D1 | Peripheral D1 |
| | bromide | | | dopamine |
| | | | | receptor agonist |
| F | SKF 83959 | 6-chloro-7,8-dihydroxy-3-methyl- | D1 | Atypical D1 |
| | hydrobromide | 1-(3-methylphenyl)-2,3,4,5- | | dopamine |
| | | tetrahydro-1H-3-benzazepine | | receptor agonist; |
| | | hydrobromide | | displays |
| | | | | antagonist activity |
| | | | | in vitro and |
| | | | | agonist activity in |
| | | | | vivo |
| G | SKF 89626 | 4-(3,4-dihydroxyphenyl)-4,5,6,7- | D1 | D1 dopamine |
| | | tetrahydrothieno[2,3-c]pyridine | | receptor agonist |
| H | (±)-SKF | (±)-7,8-Dihydroxy-3-allyl-1- | D1 | D1 dopamine |
| | 38393, N-allyl-, | phenyl-2,3,4,5-tetrahydro-1H-3- | | receptor agonist. |
| | hydrobromide | benzazepine hydrobromide | | |
| I | (±)-6-Chloro- | (±)-SKF-81297 hydrobromide | D1 | D1 dopamine |
| | PB | | | receptor agonist |
| | hydrobromide | | | |

### REFERENCES

1. Selkoe D J (1999) Translating cell biology into therapeutic advances in Alzheimer's disease. Nature Supp. 399:A23-A31.
2. Mendell J R, Sahenk Z, Gales T, Paul L (1991) Amyloid Filaments in Inclusion Body Myositis. Arch. Neurol. 48:1229-1234.
3. Kalaria R N, Ballard C (1999) Overlap Between Pathology of Alzheimer Disease and Vascular Dementia. Alzheimer Disease & Associated Disorders 13:S115-S123.
4. Klein W L, Krafft G A, Finch C E (2001) Targeting small Aβ oligomers: the solution to an Alzheimer's disease conundrum? Trends Neurosci. 24:219-224.
5. Lambert M P, Barlow A K, Chromy B A, Edwards C, Freed R, Liosatos M, Morgan T E, Rozovsky I, Trommer B, Viola K L and others (1998) Diffusible, nonfibrillar ligands derived from Aβ1-42 are potent central nervous system neurotoxins. Proc. Nat. Acad. Sci. U.S.A. 95:6448-6453.
6. Lesne S, Ming T K, Kotilinek L, Kayed R, Glabe C G, Yang A, Gallagher M, Ashe K H (2006) A specific amyloid-β protein assembly in the brain impairs memory. Nature 440:352-357.
7. Haass C, Selkoe D J (2007) Soluble protein oligomers in neurodegeneration: Lessons from the Alzheimer's amyloid β-peptide. Nat. Rev. Mol. Cell Biol. 8:101-112.
8. Walsh D M, Selkoe D J (2007) Aβ Oligomers—a decade of discovery J. Neurochem. 101:1172-1184.
9. Kebabian J W, Britton D R, DeNinno M P, Perner R, Smith L, Jenner P, Schoenleber R, Williams M (1992) A-77636: a potent and selective dopamine D1 receptor agonist with antiparkinsonian activity in marmosets. Eur. J. Pharmacol. 229:203-209.
10. Chausmer A L, Katz J L (2002) Comparison of interactions of D1-like agonists, SKF 81297, SKF 82958 and A-77636, with cocaine: locomotor activity and drug discrimination studies in rodents. Psychopharmacology 159:145-153.
11. Shearman M S, Ragan C I, Iversen L L (1994) Inhibition of PC12 cell redox activity is a specific, early indicator of the mechanism of β-amyloid mediated cell death. Proc. Nat. Acad. Sci. U.S.A. 91:1470-1474.
12. Datki Z, Juhasz A, Galfi M, Soos K, Papp R, Zadori D, Penke B (2003) Method for measuring neurotoxicity of aggregating polypeptides with the MTT assay on differentiated neuroblastoma cells. Brain Res. Bull. 62:223-229.
13. Kim H., Yoon S. C., Lee T. Y. and Jeong D. 2009. Discriminative cytotoxicity assessment based on various cellular damages. Toxicol Lett. 184:13-17.
14. Koh J. Y. and Choi D. W. 1987. Quantitative determination of glutamate mediated cortical neuronal injury in cell culture by lactate dehydrogenase efflux assay. J Neurosci. 20: 83-90.
15. Lobner D. 2000. Comparison of the LDH and MTT assays for quantifying cell death: validity for neuronal apoptosis. J Neurosci Meth. 96:147-152.
16. Kebabian J W, Britton D R, DeNinno M P, Perner R, Smith L, Jenner P, Schoenleber R, Williams M. A-77636: a potent and selective dopamine D1 receptor agonist with antiparkinsonian activity in marmosets. Eur J Pharmacol. 1992 Dec. 15; 229(2-3):203-9.
17. Chan J. N. Y., Vuckovic D., Sleno L., Olsen J. B., Pogoutse O., Havugimana P., Hewel J. A., Bajaj N., Wang Y., Musteata M. F., Nislow C. and Emili A. 2012. Target Identification by Chromatographic Co-Elution: Monitoring of drug-protein interactions without immobilization or chemical derivatization. Am Soc Biochem Mol Biol. 1-40.
18. Franekova V., Baliova M. and Jursky F. 2008. Truncation of human dopamine transporter by protease calpain. Neurochem Int. 52: 1436-1441.
19. Lan J., Skeberdis V. A., Jover T., Grooms S. Y., Lin Y., Araneda R. C., Zheng X., Bennett M. V. L. and Zukin S. R. 2001. Protein kinase C modulates NMDA receptor trafficking and gating. Nat Neurosci. 4(4): 382-390.
20. Liu W., Dou F., Feng J. and Yan Z. 2011. RACK1 is involved in β-amyloid impairment of muscarinic regulation of GAB Aergic transmission. Neurobiol Aging. 32: 1818-1826.
21. Sklan E. H., Podoly E. and Soreq H. 2006. RACK1 has the nerve to act: Structure meets function in the nervous system. Prog Neurobiol. 78: 117-134.

1. A composition for the treatment of a disease or condition associated with β-amyloid induced toxicity, comprising the compound 3-(1′-adamantyl)-1-aminomethyl-3,4-dihydro-5,6-dihydroxy-1H-2-benzopyran, or a pharmaceutically acceptable salt or solvate thereof.

2. The composition according to claim 1, wherein said compound is (1R,3S)3-(1′-adamantyl)-1-aminomethyl-3,4-dihydro-5,6-dihydroxy-1H-2-benzopyran or a pharmaceutically acceptable salt or solvate thereof.

3. The composition according to claim 2, wherein said compound is (1R,3S)3-(1′-adamantyl)-1-aminomethyl-3,4-dihydro-5,6-dihydroxy-1H-2-benzopyran hydrochloride.

4. A pharmaceutical composition for the treatment of a disease or condition associated with β-amyloid induced toxicity, comprising the compound defined in claim 1, or a pharmaceutically acceptable salt or solvate thereof, and one or more pharmaceutically acceptable excipients.

5. The composition according to claim 1, wherein the disease or condition associated with β-amyloid induced toxicity is selected from Alzheimer's disease, inclusion body myositis and vascular dementia and cerebral amyloid angiopathy.

6. The compound according to claim 1, wherein the disease or condition associated with β-amyloid induced toxicity is Alzheimer's disease.

7. A method of treating a disease or condition associated with β-amyloid induced toxicity, said method comprising administering to a subject in need of such treatment a therapeutically effective amount of the compound defined in claim 1, or a pharmaceutically acceptable salt or solvate thereof.

8. The method according to claim 7, wherein the disease or condition associated with β-amyloid induced toxicity is selected from Alzheimer's disease, inclusion body myositis and vascular dementia and cerebral amyloid angiopathy.

9. The method according to claim 7, wherein the disease or condition associated with β-amyloid induced toxicity is Alzheimer's disease.

10. (canceled)

11. A method of inhibiting β-amyloid induced toxicity (in vitro or in vivo), said method comprising administering an effective amount of 3-(1′-adamantyl)-1-aminomethyl-3,4-dihydro-5,6-dihydroxy-1H-2-benzopyran, or a pharmaceutically acceptable salt or solvate thereof.

12. The pharmaceutical composition according to claim 4, wherein the disease or condition associated with β-amyloid induced toxicity is selected from Alzheimer's disease, inclusion body myositis and vascular dementia and cerebral amyloid angiopathy.

13. The pharmaceutical composition according to claim 4, wherein the disease or condition associated with β-amyloid induced toxicity is Alzheimer's disease.

14. A method of treating a disease or condition associated with β-amyloid induced toxicity, said method comprising administering to a subject in need of such treatment a therapeutically effective amount of a pharmaceutical composition according to claim 4.

15. The method according to claim 14, wherein the disease or condition associated with β-amyloid induced toxicity is selected from Alzheimer's disease, inclusion body myositis and vascular dementia and cerebral amyloid angiopathy.

16. The method according to claim 14, wherein the disease or condition associated with β-amyloid induced toxicity is Alzheimer's disease.
